(19) United States
(12) Patent Application Publication
(10) Pub. No.: US 2025/0318899 A1
(43) Pub. Date: Oct. 16, 2025

(54) MEDICAL DEVICE WITH BEADED STRUCTURE FOR LOCALIZATION AND MARKING

(71) Applicant: Xuanwu Hospital Capital Medical University, Beijing (CN)

(72) Inventors: Kaifu LI, Beijing (CN); Hua KANG, Beijing (CN); Yang LIU, Beijing (CN)

(73) Assignee: Xuanwu Hospital Capital Medical University, Beijing (CN)

(21) Appl. No.: 19/066,709

(22) Filed: Feb. 28, 2025

Related U.S. Application Data
(63) Continuation of application No. PCT/CN2024/099209, filed on Jun. 14, 2024.

(30) Foreign Application Priority Data
Apr. 15, 2024 (CN) 202410447023.0

(51) Int. Cl. A61B 90/00

(52) U.S. Cl. CPC A61B 90/39; A61B 2090/3908; A61B 2090/3941; A61B 2090/3987; A61B 2090/3991

(57) ABSTRACT

A medical localization and marking device with a beaded structure is provided, including a long, cylindrical syringe for puncture, a marker for tumor localization, and a fixing device acting between the marker and the syringe. The marker includes a hook portion arranged at a front end of a string of beads, and the string of beads composed of a string and a plurality of beads uniformly fixed on the string and spaced apart from each other. The fixing device includes a fixing block and upper and lower shells that are mutually clamped. A contact surface of the mutually clamped upper and lower shells is provided with an elastic slot corresponding to the string of beads.

### CROSS REFERENCE TO RELATED APPLICATION

This application claims priority to Chinese Patent Application No. CN202410447023.0, filed on Apr. 15, 2024, and International Patent Application No. PCT/CN2024/099209, filed on Jun. 14, 2024, both of which are hereby incorporated by reference in their entireties.

### TECHNICAL FIELD

The present application relates to a surgical product and, in particular, to a medical localization and marking device with a beaded structure that can accurately mark the range of tumor resection, belonging to the technical field of medical supplies.

### BACKGROUND

Local extended resection of breast cancer is one of the standard surgical methods for treating early-stage breast cancer. Compared with total mastectomy, this method not only results in less surgical trauma and faster postoperative recovery but also preserves the shape of the breast while ensuring effective tumor treatment, thereby encouraging a positive psychological reaction from the patient after surgery. At present, the extent of local extended resection of breast cancer is based mainly on the results of palpation by the surgeon. For unpalpable or small-sized breast tumors, a needle with a hookwire is generally used to localize the tumor. However, this localization method cannot guarantee precise excision of the tumor and sometimes results in excessive resection of normal tissue.

In recent years, neoadjuvant chemotherapy has become increasingly common in the treatment of breast cancer. As tumors may shrink or even disappear after neoadjuvant chemotherapy, it is clinically necessary to place a metallic clip as a marker in the center or periphery of the tumor or apply skin tattoos to mark the location of the tumor in advance. Then, before breast-conserving surgery, inverted hookwire needle localization is performed under ultrasound guidance to determine the extent of tumor resection. Afterward, the surgeon finally performs the breast-conserving surgery according to the extent of skin marking and the location of the inverted hookwire needle in combination with the findings from palpation.

In the above procedure, the skin tattoos can only mark an approximate range, and so the localization may not be accurate because of relative positional changes between the glands and the skin marking points during the operation. Owing to costs or clinical convenience issues, metallic clip markers usually only mark the central part of the tumor. Even if the metallic clip markers are placed at the edge of the tumor, the markings are dotted rather than lined or planar. Moreover, preoperative placement of the inverted hookwire needle under ultrasound guidance for metallic clip marker localization not only requires repuncturing the patient but also can result in problems such as inaccurate or difficult localization in, for example, calcified lesions. In addition, the inverted hookwire needle is made of metal and may have abnormal conductivity, which can lead to some problems and unwanted effects on the surgical process.

Therefore, the main objective of the present disclosure is to design a medical localization and marking device with a beaded structure to modify existing tumor boundary marking methods and achieve “precise resection” of the tumor.

### SUMMARY

In view of the above, the present disclosure aims to provide a novel medical localization and marking device with a beaded structure that can significantly simplify the preoperative tumor localization process and facilitate the tumor resection procedure to overcome the shortcomings of existing methods including skin tattooing, metallic clip marker placement, inverted hookwire needle insertion and other localization methods, meet the requirements of clinical tumor surgery, and improve the treatment effects of breast-conserving surgery for breast cancer, especially for patients with breast cancer receiving neoadjuvant treatment.

To achieve the above objectives, the present disclosure adopts the following technical solutions.

In some embodiments of the present disclosure, a medical localization and marking device with a beaded structure is provided, which includes a long, cylindrical syringe for puncture, a marker for tumor localization, and a fixing device acting between the marker and the syringe. The opening of a needle at the head portion of the syringe is beveled, and the marker includes a string of beads and a hook portion. The hook portion is arranged at a front end of the string of beads; then, the front end of the marker is inserted into the syringe, and the rear end of the marker is exposed on the outside of the tail portion of the syringe. The string of beads is composed of a string and multiple beads uniformly fixed on the string and spaced apart from each other. The fixing device includes a fixing block, which is placed on the syringe, and upper and lower shells that are mutually clamped at the tail portion of the syringe via being limited by the fixing block. The contact surface of the mutually clamped upper and lower shells is provided with an elastic slot corresponding to the string of beads.

In some examples of the present disclosure, the elastic slot is an open slot, and a silicone or a sponge layer covers the surface of the open slot.

In some embodiments of the present disclosure, a medical localization and marking device with a beaded structure is provided, which includes a long cylindrical syringe for puncture, an inner barrel inserted into the syringe, a marker for tumor localization, and a fixing device acting between the marker and the syringe. The opening of the needle at the head portion of the syringe is beveled, and the marker includes a string of beads and a hook portion. The hook portion is arranged at the front end of the string of beads and passes through the inner barrel to be located within the syringe. The string of beads is composed of a string and multiple beads uniformly fixed on the string and spaced apart from each other. The rear end of the inner barrel protrudes outward from the tail portion of the syringe, and the front end of the inner barrel is pressed against the bottom portion of the hook portion. The fixing device includes two fixing blocks and upper and lower shells that are mutually clamped. The two fixing blocks are respectively placed on the syringe and the inner barrel, and the upper and lower shells are clamped onto the syringe and the inner barrel and limited by the two fixing blocks.

In some examples of the present disclosure, at least one of fluorescent material or developing material is distributed on the marker.

In some examples of the present disclosure, the beads are numbered in order or at intervals.

In some examples of the present disclosure, the beads are colored in order or at intervals.

In some examples of the present disclosure, the beads are round, oval, fusiform, or cylindrical.

In some examples of the present disclosure, the hook portion is cylindrical and is provided with at least one annular groove near the front end of the cylindrical hook portion.

In some examples of the present disclosure, a cavity is provided between the mutually clamped upper and lower shells for embedding and clamping the fixing block; alternatively, the mutually clamped upper and lower shells are provided with a fixing hole or fixing slot for embedding and clamping the fixing block.

The medical localization and marking device with a beaded structure in the present disclosure has at least the following beneficial effects and advantages.

1. By uniformly fixing the beads on the string and spacing them apart from each other, the string of beads can bend well, which facilitates the marking and subsequent cutting off processes. Moreover, using the heat-resistant polymeric material to produce the beads minimizes the risk of the beads being cut by a scalpel or electrotome.

2. The marker is composed of a string of beads and a hook portion, which can be easily embedded into surrounding tissues, preventing marker displacement. The simultaneous use of multiple markers can aid in marking and locating the tumor in multiple directions, providing a basis for and guaranteeing the subsequent “precise resection” of the tumors.

3. By using different numbers, colors, and shapes for the beads arranged in sequence or intervals, the length of the string of beads can be quickly determined during surgery, facilitating the surgical process and allowing the surgeon to determine whether any beads remain in the body after surgery. Moreover, by adding fluorescent materials to the marker and employing far-infrared fluorescence detectors to excite the materials, the marker can be localized, further facilitating the surgical process.

4. Setting the fixing device can facilitate the marker and syringe localization processes, meeting the requirements for clinical operations.

5. Setting the inner barrel structure can facilitate extrusion and positioning of the marker, improving the accuracy and reliability of the delivery process.

6. The overall structure of the medical localization and marking device with the beaded structure of the present disclosure is simple and easy to operate, fully meeting the needs of tumor localization and marking prior tumor resection, and is especially suitable for tumor localization and marking for patients receiving neoadjuvant therapy and planned breast-conserving surgery.

### BRIEF DESCRIPTION OF THE DRAWINGS

To provide a clearer explanation of the technical solutions in the embodiments of the present disclosure, the following will briefly introduce the accompanying drawings required for the description of the embodiments of the present disclosure. It is obvious that the accompanying drawings described below are only some embodiments of the present disclosure. For one with ordinary skill in the art, other drawings may be obtained on the basis of these drawings without creative labor.

FIG. 1 shows a schematic diagram of an assembly structure of a medical localization and marking device with a beaded structure in accordance with some embodiments of the present disclosure.

FIG. 2 shows a second schematic diagram of the assembly structure of the medical localization and marking device with a beaded structure in accordance with some embodiments of the present disclosure.

FIG. 3 shows a partially enlarged sectional view of FIG. 2 at point A.

FIG. 4 shows a third schematic diagram of the assembly structure of the medical localization and marking device with a beaded structure in accordance with some embodiments of the present disclosure.

FIG. 5 shows a fourth schematic diagram of the assembly structure of the medical localization and marking device with a beaded structure in accordance with some embodiments of the present disclosure.

### DETAILED DESCRIPTION

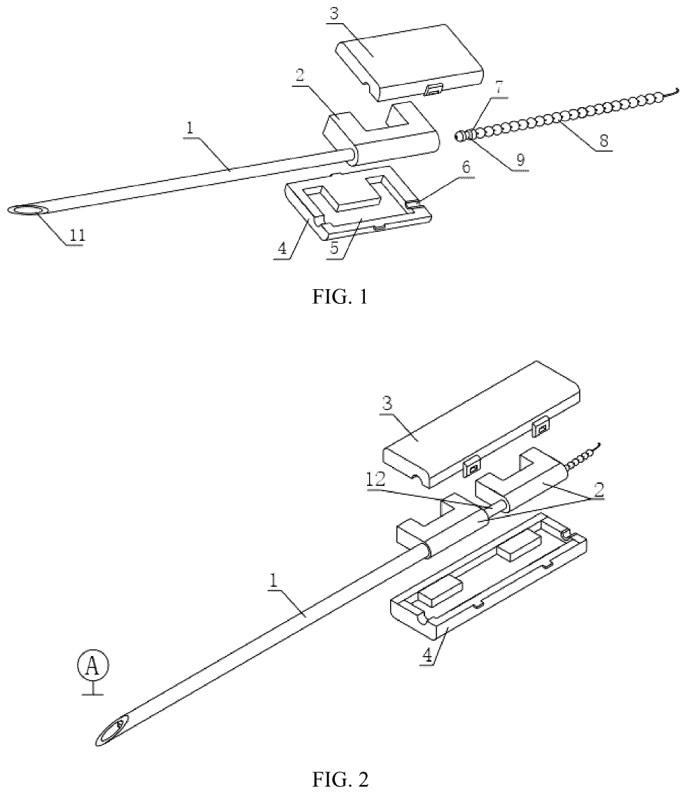

The present disclosure will be further described in detail with reference to FIG. 1.

In some embodiments of the present disclosure, a medical localization and marking device with a beaded structure is provided, including a long cylindrical syringe 1 for puncture, a marker for tumor localization, and a fixing device acting between the marker and the syringe 1.

The head portion of the long cylindrical syringe 1 used for puncture is needle shaped. For the convenience of the puncture process and subsequent rapid hooking and localization of the marker to the patient's tissue, the opening 11 of the needle is beveled, facilitating observation and manipulation of the marker to complete the hooking operation.

The marker includes a string of beads 8 and a hook portion 7 arranged at a front end of the string of beads 8. The string of beads 8 is composed of a string and multiple beads uniformly fixed on the string and spaced apart from each other. In some examples, the beads are round and made of heat-resistant polymeric material. The beads may also be oval, fusiform, or cylindrical. After the front end of the marker is inserted into syringe 1, the rear end of the marker is exposed on the outside of the tail portion of syringe 1. To facilitate searching for the position of the marker during surgery, in some examples, fluorescent materials are mixed onto the marker, and a far-infrared detector can be used to assist in detecting the marker position. To further facilitate the surgical process, in some examples, the beads are colored at fixed intervals, and the length of the string of beads can be determined by the color and the number of beads at these fixed intervals. Different shapes of beads can also be arranged at fixed intervals, and codes can be directly set onto the beads in a sequential or interval arrangement, allowing easy determination of the length of the string of beads 8 during surgery. Hook portion 7 is cylindrical overall and is provided with an annular groove 9 near the front end of this cylindrical hook portion 7. The annular groove 9 can be used to attach the hook portion 7 to the patient's tissue, and the string of beads 8 connected behind it can also be embedded in the patient's tissue and fixed in position, thereby allowing multidimensional positioning and marking of the location of the tumor through the attachment of multiple markers.

The fixing device includes fixing block 2, and upper and lower shells 3 and 4 that are mutually clamped. In some examples, the fixing block 2 is in the shape of a “]” and is placed at the tail portion of the syringe 1. A cavity 5 is provided between the upper and lower shells 3 and 4 which are mutually clamped, for embedding and clamping the “]”-shaped fixing block 2. Specifically, the upper and lower shells 3 and 4 are clamped at the tail portion of the syringe 1 via being limited by the fixing block 2. The contact surface of the mutually clamped upper and lower shells 3 and 4 is provided with an elastic slot 6 through which the string of beads can be fed. In some examples of the present disclosure, the elastic slot 6 is an open slot, and a silicone layer covers the surface of the open slot. After being limited, the upper and lower shells 3 and 4 can clamp the exposed string of beads 8 through deformation of the silicone layer, thereby forming a limit between the string of beads 8 and the syringe 1. Notably, the fixing block 2 can also be distributed on both lateral sides of the syringe 1, and the upper and lower shells 3 and 4 may also be provided with a fixing hole or a fixing slot for fixing with the fixing block 2.

Prior to a specific marking operation, first, the string of beads 8 is pushed inside the syringe 1 so that the hook portion 7 at the front end of the string of beads 8 ends up close to the beveled opening 11 of the needle at the front end of the syringe 1; then, the upper and lower shells 3 and 4 wrap and embed the fixing block 2 into the clamping cavity 5. Finally, the upper and lower shells 3 and 4 are clamped together to complete the limit connection of the upper and lower shells 3 and 4 at the tail portion of the syringe 1. Simultaneously, the elastic slot 6 clamps the beads 8 exposed outside the fixing block 2, completing the limit between the syringe 1 and the marker.

When performing the specific marking operation, the patient's tissue is punctured with the syringe 1, whose position can be observed through imaging modalities such as ultrasound and mammography. After the head portion of the syringe 1 reaches the target, the upper and lower shells 3 and 4 are separated, and the string of beads 8 are pushed to allow the hook portion 7 to extend from the opening 11 of the needle. After the annular groove 9 on the hook portion 7 is attached to the patent's tissue, the string of beads 8 are pushed inward, while the syringe 1 is pulled outward to exit the patient's body. The marker is left in the patient's body, and the part of the string of beads 8 exposed outside the patient's body is cut off. Different colored beads at fixed intervals can help to quickly determine the length of the part of the string of beads 8 remaining in the patient's body, thus completing the marking process.

After performing such operations several times, the patient's tumor can be located and marked in multiple directions with strings of beads 8 of multiple markers. The color difference between the marker and human tissue can be used to observe and indicate the position of the marker at any time. If necessary, far-infrared fluorescence probes can be used to detect the fluorescent material mixed in the marker, indicating the marking location, providing convenience and assistance for accurate tumor resection on the basis of the range of the beaded marker in the future, and improving the efficiency of precise tumor resection.

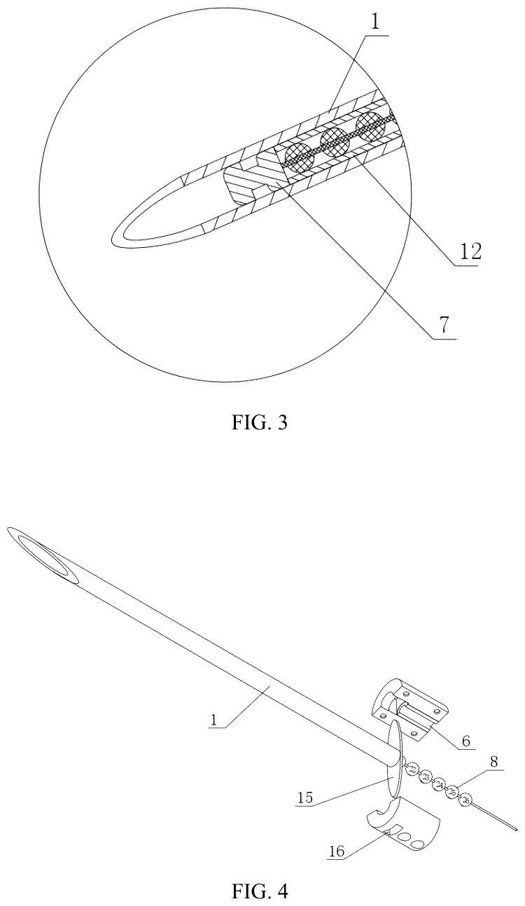

The above description involves only some specific implementations, as shown in FIG. 2 and FIG. 3. In some other examples of the present disclosure, a medical localization and marking device with a beaded structure is provided, including a long cylindrical syringe 1 for puncture, an inner barrel 12 inserted into the syringe 1, a marker for tumor localization, and a fixing device acting between the inner barrel 12 and the syringe 1. In this example of the present disclosure, the syringe 1 and the marker have the same structure as the syringe 1 and the marker, respectively, described above. Additionally, in this example of the present disclosure, the inner barrel 12 is inserted into the syringe 1 to push the marker, and the rear end of the inner barrel 12 protrudes outward from the tail portion of the syringe 1. After the marker passes through the inner barrel 12, the front end of the inner barrel 12 is pressed against the bottom portion of the hook portion 7 at the front end of the marker to simplify insertion and attachment of the marker, thereby improving the convenience and flexibility of the pushing process. At the same time, to simplify the limiting process, the fixing device includes two “]” shaped fixing blocks 2, which are set on the syringe 1 and the inner barrel 12 respectively. The upper and lower shells 3 and 4 are mutually clamped onto the syringe 1 and the inner barrel 12, surrounding the two fixing blocks 2, thereby limiting the movement between the inner barrel 12 and the syringe 1. Because the front end of the inner barrel 12 is pressed against the bottom portion of the hook portion 7 at the front end of the marker, the marker is also limited from moving within the syringe 1.

### Embodiment 1

Referring to FIG. 4, in some examples of the present disclosure, a medical localization and marking device with a beaded structure is provided, including a long, cylindrical syringe 1 for puncture, a marker for tumor localization, and a fixing device acting between the marker and the syringe 1. The head portion of the syringe 1 is needle-like, and the opening of the needle is beveled. The marker includes a string of beads 8 and a hook portion. The string of beads 8 is composed of a string and a plurality of beads uniformly fixed on the string and spaced apart from each other. To provide visual feedback on the position information of the string of beads 8, in some examples, each bead is sequentially numbered. The hook portion is arranged at the front end of the string of beads 8. The front end of the marker is inserted into the syringe 1, and the rear end of the marker is exposed on the outside of the tail portion of the syringe 1. The fixing device includes a sheet-like fixing block 15 and upper and lower shells that are mutually clamped. The sheet-like fixing block 15 is integrated with the syringe 1. The sheet-like fixing block 15 is inserted into the fixing hole 16 of the upper and lower shells, and then through the mutual clamping of the upper and lower shells, the limiting connection between the upper and lower shells and the syringe 1 is completed. The contact surface of the mutually clamped upper and lower shells is provided with the elastic slot 6 corresponding to the string of beads 8, and the elastic slot 6 allows the positions of the string of beads 8 and the syringe 1 to be fixed relative to each other.

Before the specific marking operation, the hook portion located at the front end of the marker in the syringe 1 should be placed as close as possible to the opening of the needle. The marker is then clamped by the upper and lower shells, fixing its position within the syringe 1. When performing the specific marking operation, an imaging device is used to guide the puncture with the syringe 1. After the tip of the needle reaches the target, the upper and lower shells are opened, allowing the string of beads 8 to be pushed directly and extend the hook portion forward. After the hook portion connects with the patient's tissue, the string of beads 8 is pushed inward while syringe 1 is pulled outward, leaving the marker in the patient's body. Any excess beads exposed outside the patient's body are removed to complete multidirectional marking and localization of the periphery of the tumor.

### Embodiment 2

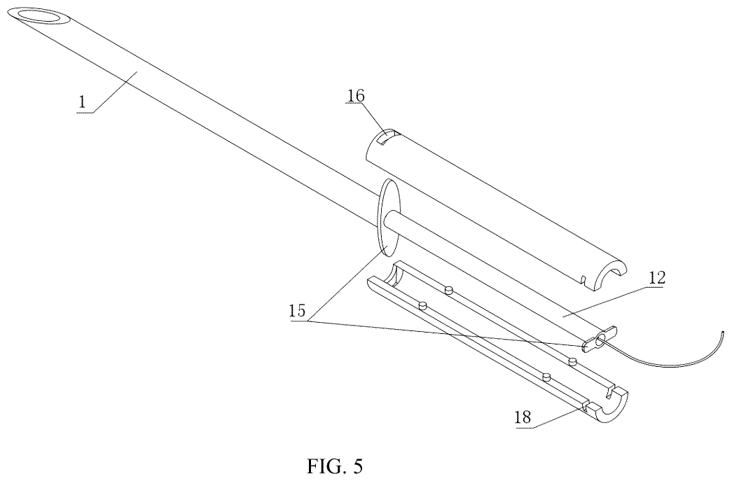

Referring to FIG. 5, in some examples of the present disclosure, a medical localization and marking device with a beaded structure is provided, including a long, cylindrical syringe 1 for puncture, an inner barrel 12 inserted into the syringe 1, a marker for tumor localization, and a fixing device acting between the inner barrel 12 and the syringe 1. Herein, the head portion of the syringe 1 is needle-like, and the opening of the needle is beveled. The rear end of the inner barrel 12 protrudes outward from the tail portion of the syringe 1. After the marker passes through the inner barrel 12, the front end of the inner barrel 12 is pressed against the bottom portion of the hook portion 7 at the front end of the marker. To limit movement between the inner barrel 12 and the syringe 1, a fixing device including two sheet-like fixing blocks 15 and upper and lower shells that are mutually clamped are positioned. The two sheet-like fixing blocks 15 are set at the tail portion of the syringe 1 and the tail portion of the inner barrel 12 respectively. Each of the mutually clamped upper and lower shells is provided with a fixing hole 16 and a fixing slot 18 that correspond to the two sheet-like fixing blocks 15 respectively for clamping. The relative position between the inner barrel 12 and the syringe 1 is fixed by the limit of the sheet-like fixing blocks 15 and the mutual clamping of the upper and lower shells.

Before the specific marking operation, the position of the inner barrel 12 is fixed within syringe 1, and the front end of the inner barrel 12 is pressed against the bottom portion of the hook portion, relatively fixing the position of the marker in the inner barrel 12 and the syringe 1. When performing the specific marking operation, the patient's tissue is accurately punctured with the syringe 1; after the tip of the needle reaches the target, the upper and lower shells are opened, allowing the inner barrel 12 to be pushed to drive the marker forward, completing the attachment between the hook portion and the patient's tissue. The marker is pushed inward while retracting the syringe 1 and the inner barrel 12 from the patient's body, and any excess beads exposed outside the patient's body are removed. The marker now allows multidimensional localization and marking of the tumor inside the patient's body.

The above description only applies to the preferred embodiments of the present disclosure and the explanation of the technical principles applied and is not intended to limit the scope of protection of the present invention but only represents the preferred embodiments of the present disclosure. One of ordinary skill in the art should understand that the invention scope of the present disclosure is not fixed to the technical solutions formed by a specific combination of the above technical features and should also cover other technical solutions formed by arbitrary combinations of the above technical features or their equivalent features without departing from the inventive concept. On the basis of the embodiments of the present disclosure, all other embodiments obtained by those skilled in the art without creative labor are within the scope of protection of the present invention.

What is claimed is:

1. A medical localization and marking device with a beaded structure, comprising a long, cylindrical syringe for puncture, a marker for tumor localization, and a fixing device acting between the marker and the syringe; wherein an opening of a needle at a head portion of the syringe is beveled; wherein the marker comprises a string of beads and a hook portion arranged at a front end of the string of beads, a front end of the marker is inserted into the syringe, and a rear end of the marker is exposed on outside of a tail portion of the syringe; wherein the string of beads is composed of a string and a plurality of beads uniformly fixed on the string and spaced apart from each other; wherein the fixing device comprises a fixing block and upper and lower shells that are mutually clamped; wherein the fixing block is placed on the syringe, the upper and lower shells are clamped at the tail portion of the syringe via being limited by the fixing block, and a contact surface of the mutually clamped upper and lower shells is provided with an elastic slot corresponding to the string of beads.

2. The medical localization and marking device with the beaded structure of claim 1, wherein the elastic slot is an open slot, and a silicone layer or a sponge layer covers a surface of the open slot.

3. The medical localization and marking device with the beaded structure of claim 1, wherein at least one of fluorescent material or developing material is distributed on the marker.

4. The medical localization and marking device with the beaded structure of claim 1, wherein the plurality of beads are numbered in order or at intervals.

5. The medical localization and marking device with the beaded structure of claim 1, wherein the plurality of beads are colored in order or at intervals.

6. The medical localization and marking device with the beaded structure of claim 1, wherein the plurality of beads are round, oval, fusiform, or cylindrical.

7. The medical localization and marking device with the beaded structure of claim 1, wherein the plurality of beads are made of heat-resistant polymeric material.

8. The medical localization and marking device with the beaded structure of claim 1, where the hook portion is cylindrical and is provided with at least one annular groove near a front end of the cylindrical hook portion.

9. The medical localization and marking device with the beaded structure of claim 1, wherein a cavity is provided between the mutually clamped upper and lower shells for embedding and clamping the fixing block.

10. The medical localization and marking device with the beaded structure of claim 1, wherein the mutually clamped upper and lower shells are provided with a fixing hole or fixing slot for embedding and clamping the fixing block.

11. A medical localization and marking device with a beaded structure, comprising a long cylindrical syringe for puncture, an inner barrel inserted into the syringe, a marker for tumor localization, and a fixing device acting between the marker and the syringe; wherein an opening of a needle at a head portion of the syringe is beveled; wherein the marker comprises a string of beads and a hook portion, and the hook portion is arranged at a front end of the string of beads and passes through the inner barrel to be located within the syringe; wherein the string of beads is composed of a string and a plurality of beads uniformly fixed on the string and spaced apart from each other; wherein a rear end of the inner barrel protrudes outward from a tail portion of the syringe, and a front end of the inner barrel is pressed against a bottom portion of the hook portion; and wherein the fixing device comprises two fixing blocks, respectively placed on the syringe and the inner barrel, and upper and lower shells that are mutually clamped onto the syringe and the inner barrel and limited by the two fixing blocks.

12. The medical localization and marking device with the beaded structure of claim 11, wherein at least one of fluorescent material or developing material is distributed on the marker.

13. The medical localization and marking device with the beaded structure of claim 11, wherein the plurality of beads are numbered in order or at intervals.

14. The medical localization and marking device with the beaded structure of claim 11, wherein the plurality of beads are colored in order or at intervals.

15. The medical localization and marking device with the beaded structure of claim 11, wherein the plurality of beads are round, oval, fusiform, or cylindrical.

16. The medical localization and marking device with the beaded structure of claim 11, wherein the plurality of beads are made of heat-resistant polymeric material.

17. The medical localization and marking device with the beaded structure of claim 11, wherein the hook portion is cylindrical and is provided with at least one annular groove near a front end of the cylindrical hook portion.

18. The medical localization and marking device with the beaded structure of claim 11, wherein a cavity is provided between the mutually clamped upper and lower shells for embedding and clamping the two fixing blocks.

19. The medical localization and marking device with the beaded structure of claim 11, wherein the mutually clamped upper and lower shells are provided with a fixing hole or fixing slot for embedding and clamping the two fixing blocks.
